(19) United States
(12) Patent Application Publication
(10) Pub. No.: US 2009/0276940 A1
(43) Pub. Date: Nov. 12, 2009

(54) Mask defogging system

(76) Inventor: Matthew Blair Sallee, Leroy, MI (US)

Correspondence Address: Matthew B. Sallee, 14492 Lakola Rd., Leroy, MI, 49655, US

(21) Appl. No.: 12/151,574

(22) Filed: May 8, 2008

(51) Int. Cl. A61F 9/02; A42B 1/06

(52) U.S. Cl. 2/435; 2/410

(57) ABSTRACT

A system for supplying and carrying air into the interior lens of a closed mask, helmet or goggle for the purpose of removing accumulated precipitation from the mask lens consisting of a battery powered air pump or aerator of any size or type which removably attaches to a person or a mask, helmet or goggle by a clip or other type of fastener which may be carried within a vest, belt pouch or on a belt pouch or on a belt or other form of carrying device or casing, or which may be build in as an integral part of a mask, helmet or goggle whereby air is carried from said pump through a single or multiple lines(s) of flexible plastic, vinyl or other type of flexible tubing or molded tubing which can be detached, or broken into two pieces, by a disconnect piece/connector (a quick disconnect piece) and whereby said tubing communicates between the pump outlet and the interior of the mask, helmet or goggle and air is directed onto the lens of the mask, helmet or goggle through some type of air inlet adapter, or air director piece, which is attached to the tubing for the purpose of directing the flow of air directly onto the interior lens of a mask, helmet or goggle for the purpose of dissipating or fully eradicating precipitation, or fog, as it is commonly called, from said lens and creating a clear view through said lens.

### BACKGROUND OF THE INVENTION

The history of mask defogging systems is limited almost exclusively to a certain type of fan unit which is placed within a paintball mask (goggle) for the purpose of removing the accumulated precipitation, or “fog”, therein. Such a system, however, presents a safety risk because it limits a person's natural ability to fully hear what is going on around them because the unit is operating and creating noise in very close proximity to the ears, which leads to the inconvenience of having to turn the fan unit on and off throughout play or adapt to the noise.

In addition, said fan units merely circulate the warm, humid air near the top of the head and face and fans it down into the mask, whereby this new defogging system pulls outside air in and forces a more concentrated air stream directly onto the lens of a mask, helmet or goggle, not merely fanning the air around. Similar concepts have been invented, but their scope and purpose were for radically different issues, such as debris removal within an open helmet (air wash systems) which could never have been conceived of or used for precipitation removal, or they are fans made to circulate air which does not use air psi or liters per minute, so aside from some visual and elemental similarities, this system is an innovative and practical method designed specifically for removal of precipitation from the interior lens of a closed mask, helmet or goggle.

Furthermore, most concepts in the prior art were made for specific mask, helmet or goggle designs and dependent upon such, whereby this system is independent of any particular mask, helmet or goggle design, and with the quick disconnect feature, can be easily moved from any mask, helmet or goggle to another adding a distinct level of versatility. A solid need exists to provide a practical and versatile system which both effectively deals with the problem of accumulated precipitation, or fog, on the interior of a mask, helmet or goggle lens and does not noticeably diminish a persons ability to hear what is occurring around them.

### SUMMARY OF THE INVENTION

In regard to mask, helmet or goggle lenses, particularly masks, helmets or goggles used in the sport of paintball, in deference to the common problem of accumulated precipitation on the interior of a mask lens, commonly known as fog, which both prohibits a clear view of a person's surroundings and poses a potential safety threat as a mask must be removed fully or partially in order to wipe the interior of the lens free from precipitation, thereby exposing the face and head to possible serious injury. Moreover, to add an element of safety by creating a quieter system of fog removal than other products similar to this invention because any noise from the operation of the pump is away from the ears, or when built into or fastened onto a mask or helmet is buffered by the mask or helmet and further buffered by the addition of soundproofing material, thereby not hindering a person's ability to hear what is happening around them.

The general purpose of this invention is to create an innovative, yet simple new system for removing the “fog” or precipitation that commonly accumulates on the interior of a mask, helmet or goggle lens, as well as to add an extra level of safety to the sport of paintball, or any activity, whereby the head may be routinely exposed for the purpose of wiping the lens clean or a person's hearing may be hindered by the noise of a fan unit close to the ears of a person.

Moreover, to provide a versatile system independent of any specific mask, helmet or goggle design which can be quickly removed from one goggle, helmet or mask and easily used with another. Also, to create a solution to the current method of precipitation removal, which is a fan, which does not use psi and has no measurable air flow, and which simply fans the air around a person's head and face back into the mask.

This invention is unlike anything currently available and consists of an unparalleled system which utilizes a battery powered air pump or aerator to carry air into a mask, helmet or goggle and blow forced air directly onto the interior lens of a mask, helmet or goggle. This invention can be used continuously or discretionary as needed and provides an extra level of safety and practicality to the game of paintball or any associated activity.

This system could be used in other activities, sports or professions where foggy lenses are an issue, even being adapted to be used with pressurized forced air systems, such as in firefighting or scuba diving, or could be fitted with air filtering devices and used in professions such as exterminating whereby the outside air must first be filtered before being used to clear the lens of a mask, helmet or goggle.

Heating or cooling elements could also be added if required for a specific application.

This invention substantially differs from any other designs or concepts in the prior art in several ways:
1. This system, when used as a remote unit,gets the noise away from the ears of a person and therefore does not hinder their natural ability to hear what is occurring around them and if fastened to or built into a mask or helmet, the noise is buffered by the mask or helmet and the addition of a soundproofing material thereby creating an added level of safety to the game of paintball, or other activity.
2. This system utilizes a separate battery powered air pump or aerator which employs appropriate psi and liters per minute technology to force air onto a goggle, mask or helmet lens and which removeably affixes to a person or mask, helmet or goggle or which can be built into a mask, helmet or goggle or can be carried on a vest, belt or in a belt pouch or some other type of casing which carries air through a flexible tube or tubes, airline(s) into a mask, helmet or goggle and blows said air directly onto the mask, helmet or goggle lens to clear accumulated precipitation, or fog as it is commonly called.
3. This system employs flexible tubing to carry air from an air pump or aerator into the interior of the mask, helmet or goggle thereby not limiting a persons free movement while wearing the unit and tubing can be adapted and customized for any application.
4. This system can be easily modified or adapted to be used with any mask, helmet or
5. goggle in any sport, profession or activity, or can be designed to be built into a specific mask, helmet or goggle as an integral part of mask, helmet or goggle.
6. This system is versatile and can be worn and used in several different ways.
7. This system employs a quick disconnect piece/connector piece which allows the airline of the unit to be quickly disconnected for maintenance, part replacement or to allow the unit to be quickly changed between different masks, goggles or helmets.
8. This system can employ several different pump types and styles, any variety or combination of flexible tubing for the airline and can be adapted for use with pressurized or forced air systems as the air supply or can be outfitted with air filtering devices when outside air must be filtered or can be adapted to heat or cool air as well.
9. This system has an airflow director or air inlet director piece which forces a concentrated stream of air directly onto the interior lens or a mask, helmet or goggle and said piece can be modified for different applications as needed.
10. This system is made for the removal of precipitation from the interior lens of a closed mask, helmet or goggle and is not an airwash system meant for debris removal.
11. The battery source and pump are together in any of the shown embodiments (drawings) thereby creating a smaller, more compact unit.

Said invention can be made with different sizes, styles and types of battery powered air pumps or aerators, different types or combinations of flexible tubing of any material, different placement and configuration and multiples of said tubing, may be placed anywhere on the body or mask, can be encased in any variety of housings, can employ any type of disconnect piece/connector whereby the airline, or flexible tubing, can be detached or broken into two pieces, with any variety or type of air inlet/outlet, or airflow director piece, for the purpose of directing airflow directly onto the interior of the lens of a closed mask, helmet or goggle for the purpose of clearing it of accumulated precipitation, and that any such modifications would be obvious extensions of the basic concept of this innovative new system, which is to carry air through a flexible airline into the interior lens of a closed mask, helmet or goggle in order to clear it of precipitation, or fog as it is commonly called.

### BRIEF DESCRIPTION OF THE SEVERAL VIEWS OF THE DRAWING

FIG. 1 is a side view of my mask defogging system attached to a helmet.

FIG. 2 is the unit not shown with a helmet.

FIG. 3 is an overhead view of the airline, or tubing which carries air, if built into a helmet as an integral part of a helmet shown with the disconnect piece.

FIG. 4 is an interior view of the mask defogging system if built into a helmet as an integral part of a helmet design shown with the disconnect piece, or quick disconnect, and the air inlet piece, or airflow director which directs the air onto the interior lens of the helmet.

FIG. 5 is how I picture the unit used as a full helmet or attached to a helmet.

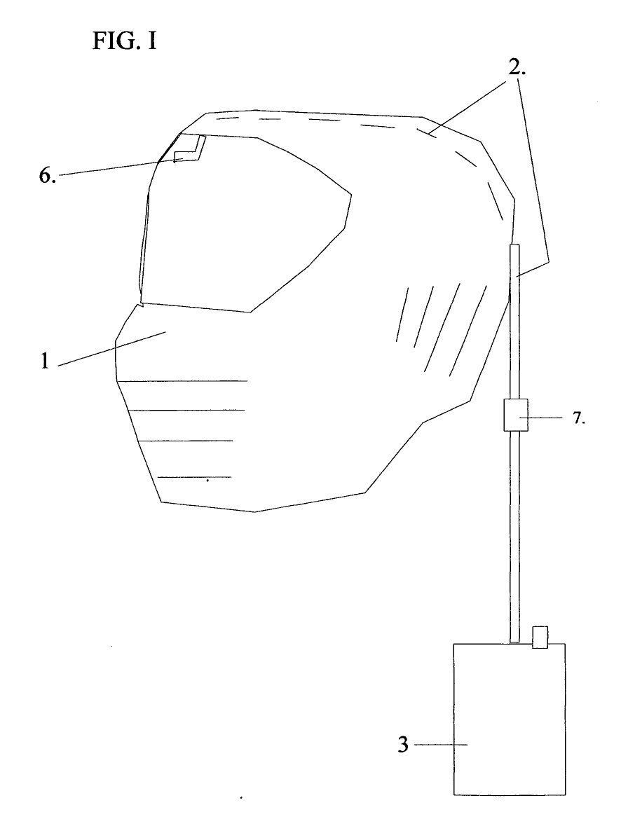

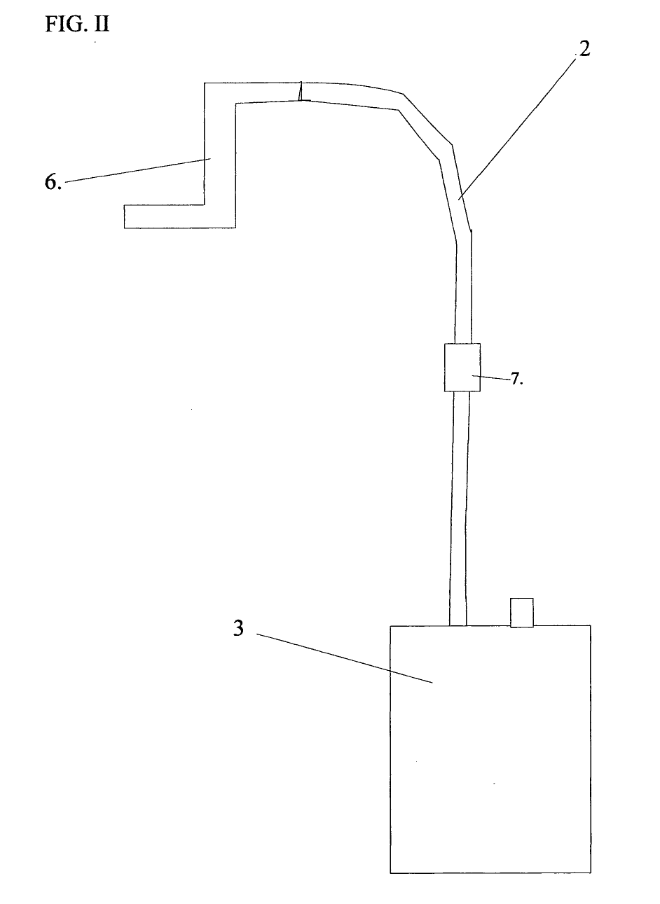

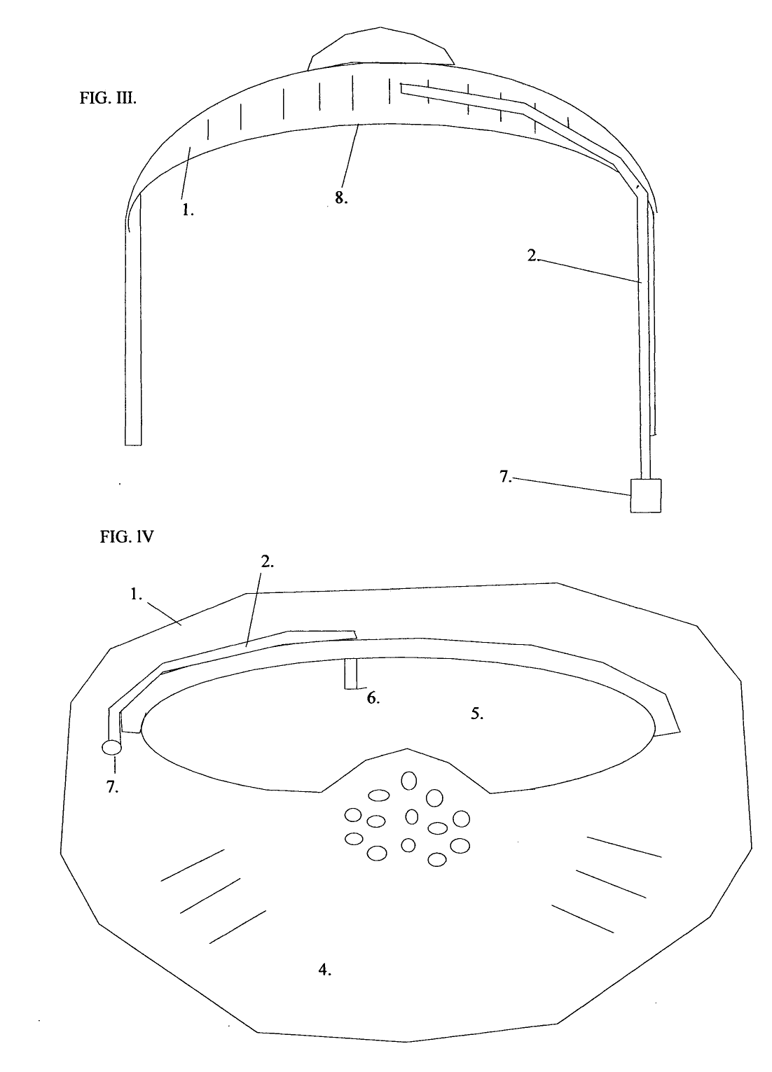

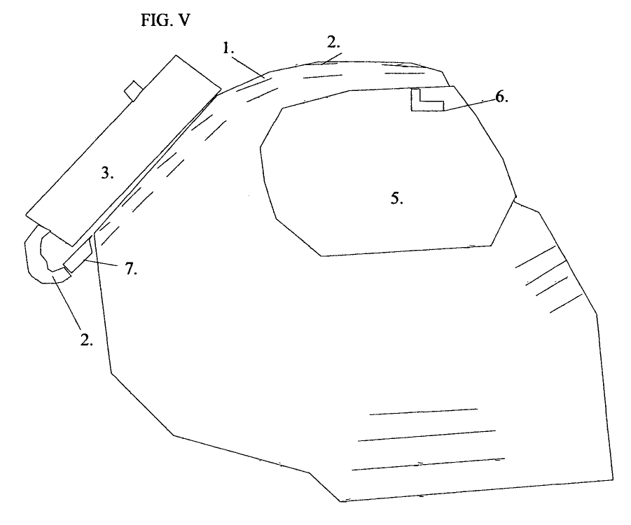

### DETAILED DESCRIPTION OF THE INVENTION

Referring to Figure I, the mask defogging system to be used with masks, helmets or goggles, particularly a paintball mask, employs a single or multiple flexible tube (2) of plastic or other flexible material for the purpose of carrying air into the interior of the mask (1) from any type of battery powered air pump or aerator (3) which is removably secured to a person by a clip or fastener whereby the end of tube which communicates with the interior of the mask ends in an air inlet piece, or airflow director (6) for the purpose of directing air directly onto the face of the lens, and dissipating or eradicating accumulated precipitation on the lens.

Referring to Figure II, a close-up of the mask defogging system to be used with masks, helmets or goggles, particularly a paintball mask, employs a battery operated air pump or aerator (3) which carries air through a single or multiple flexible tube or tubes (2) made of plastic, vinyl or other type of flexible material to create an airline by which air is carried into the interior of a mask, helmet or goggle whereby the tube employs a disconnect piece of some kind (7) for the purpose of removably discretionary detaching the air tube of which the end of the air tube which communicates between the air pump or aerator and the interior of the mask, helmet or goggle ends in an inlet tube of some kind (6) for the purpose of directing air onto the face of the lens and dissipating or eradicating accumulated precipitation.

Referring to Figure III, an overhead view (8) of the airline or tubing (2) used in the mask defogging system as a built in or integral part of a mask, helmet or goggle (1), shown with the quick disconnect piece (7) employed for the purpose of quickly disconnecting the unit if necessary.

Referring to Figure IV, an interior view (4) of the mask defogging system as a built in or integral part of a mask, helmet or goggle (1), whereby the air tube, or airline (2) is built into the mask, helmet or goggle itself, shown with the disconnect piece (7) and the air inlet piece, or airflow director (6) which directs the airflow onto the lens (6) of the mask, helmet or goggle.

Referring to Figure V, a full head cover mask or helmet (1) with the mask defogging unit designed to either fit upon the mask or helmet or be built within the mask or helmet as an integral part of the mask or helmet, showing the air pump or aerator (3) attached to a mask or helmet with the air tube, or airline (2), the disconnect piece (7) and a built-in airline (2) ending in an air inlet piece, or air director (6) directing air onto the interior lens (5) of the mask, helmet or goggle.

What I claim in my invention is:

I. A mask defogging system for supplying and carrying air into the interior of a closed mask, helmet or goggle lens to remove accumulated precipitation from said lens comprised of:
a) a battery powered air pump or aerator of any size or type which removeably attaches to a person or a mask, helmet or goggle by a clip or other type of fastener or which may be carried within a vest, belt pouch or on a belt or other form of carrying device or casing, or which may be built in as an integral part of a mask, helmet or goggle whereby air is carried from said pump through a single or multiple line(s) of flexible plastic, vinyl or other type of flexible tubing or molded tubing which can be detached, or broken into two pieces, by a disconnect piece/connector, and whereby said tubing communicates between the pump outlet and the interior of a mask, helmet or goggle and is directed onto the interior lens of the mask, helmet or goggle through some type of air inlet adapter, or air director piece, which is attached to the tubing for the purpose of directing the flow of air directly onto the interior of the lens of a mask, helmet or goggle for the purpose of dissipating or fully eradicating accumulated precipitation, or fog as it commonly called, from lens and creating a clear view through said lens.

II. It is claimed that this system is versatile in that it is detachable and can be detached quickly and easily due to the innovative quick disconnect/connector piece and may be used with any mask or goggle and is not specific to a certain mask design. Moreover, this unit can be worn either as a separate, removeably attached unit worn somewhere on a person's body or helmet, or can be carried on the body of a person in a vest, belt or in a belt pouch or casing or could be built in to a mask or helmet as an integral part of a mask or helmet and that regardless of how it is reconfigured or used, the basic concept and method of the system remains static.
